(19) United States
(12) Patent Application Publication
(10) Pub. No.: US 2026/0058001 A1
(43) Pub. Date: Feb. 26, 2026

(54) EXTRACTING FEATURES TO COMPRESS IMAGES

(71) Applicant: Intuitive Surgical Operations, Inc., Sunnyvale, CA (US)

(72) Inventors: Sreeram Kamabattula, Cumming, GA (US); Michelle Liu, Morrisville, NC (US); Conor Perreault, Atlanta, GA (US); Ziheng Wang, Atlanta, GA (US); Aneeq Zia, Alpharetta, GA (US)

(73) Assignee: Intuitive Surgical Operations, Inc., Sunnyvale, CA (US)

(21) Appl. No.: 19/304,057

(22) Filed: Aug. 19, 2025

Related U.S. Application Data
(60) Provisional application No. 63/685,072, filed on Aug. 20, 2024.

(51) Int. Cl. G16H 30/20; A61B 90/00; G06T 11/60; G06V 10/44; G16H 30/40

(52) U.S. Cl. CPC G16H 30/20; A61B 90/361; G06T 11/60; G06V 10/44; G16H 30/40

(57) ABSTRACT

Extracting features to compress images generated during medical procedures is provided. In examples, systems are configured to obtain one or more frames of a video captured by a camera of a medical procedure performed with a robotic medical system. Systems can be configured to generate features for the one or more frames using a first model trained with self-supervised machine learning and constructing a dataset based on the generated features. Some systems can be configured to construct a dataset based on the generated features and input the dataset into a second model to detect an aspect of the medical procedure.

### CROSS-REFERENCES TO RELATED APPLICATIONS

This application claims the benefit of priority under 35 U.S.C. § 119 to U.S. Provisional Patent Application No. 63/685,072, filed Aug. 20, 2024, which is hereby incorporated by reference herein in its entirety.

### BACKGROUND

During teleoperation of robotic systems, images or sets of images (e.g., a video) of one or more operations performed by the robotic system can be generated and retained for future processing. For example, in the context of robot-assisted surgical procedures, instruments can be teleoperated by a clinician (e.g., a surgeon) during one or more phases of a medical procedure, and corresponding images can be generated to provide the clinician with a view of the instruments during the procedure. The images can then be stored for later review by clinicians or researchers, for example. But recent improvements to imaging technologies have resulted in increasingly larger amounts of computing, memory and networking resources being consumed when generating and storing these images. The generation of such images can contribute to significant increases in latency as they are later processed. Further, the amount of disk space consumed when storing the images can increase as the resolution of imaging devices improves. This can make use of such images outside of well-resourced datacenters impractical.

### SUMMARY

Technical solutions disclosed herein are generally related to systems and methods for extracting features to compress images generated during medical procedures. These solutions can involve obtaining one or more frames of a video captured by a camera of a medical procedure performed with a robotic medical system. Solutions can include generating features for the one or more frames using a first model trained with self-supervised machine learning and constructing a dataset based on the generated features. Some solutions can include constructing a dataset based on the generated features and inputting the dataset into a second model to detect an aspect of the medical procedure.

Aspects of the technical solution are directed to a system. The system can include one or more processors, coupled with memory. The one or more processors can be configured to obtain one or more frames of a video captured by a camera of a medical procedure performed with a robotic medical system. The one or more processors can generate, using a first model trained with self-supervised machine learning, features for the one or more frames. The one or more processors can construct a dataset based on the generated features. The one or more processors can input the dataset into a second model to detect an aspect of the medical procedure.

In some aspects, the one or more processors can be further configured to select the second model from a plurality of second models based on an attribute of the first model.

In some aspects, the one or more processors can be further configured to determine the first model is trained with self-supervised machine learning on a type of dataset; and select the second model based on the second model being trained on the type of dataset.

In some aspects, the one or more processors can be further configured to select the first model configured to extract features based on a characteristic of the medical procedure.

In some aspects, the one or more processors can be further configured to select the second model configured to detect aspects of the medical procedure based on a characteristic of the medical procedure.

In some aspects, the one or more processors can be further configured to sample the video using a frame rate; and select the one or more frames from the sampled video.

In some aspects, the one or more processors can be further configured to select the frame rate based on a characteristic of the first model or a characteristic of the second model.

In some aspects, the one or more processors can be further configured to receive, via an interface, a request to detect a type of aspect of the medical procedure; and select the frame rate based on the type of aspect.

In some aspects, the one or more processors can be further configured to receive, via an interface, a request to detect a type of aspect of the medical procedure; and select the first model from a plurality of first models based on the type of aspect.

In some aspects, the one or more processors can be further configured to receive, via an interface, a request to detect a type of aspect of the medical procedure; and select the second model from a plurality of second models based on the type of aspect.

In some aspects, the type of aspect includes at least one of an anatomical structure, a milestone, a phase of the medical procedure, or a task of the medical procedure.

Aspects of the technical solution are directed to a system. The system can include one or more processors, coupled with memory. The one or more processors can be configured to obtain data associated with a set of images generated by at least one camera during a medical procedure; sample the set of images to generate a sampled set of images; provide an image of the sampled set of images as input to a model to cause the model to generate an output including one or more embeddings that represent one or more features, the one or more features corresponding to aspects of medical procedures in a latent space; generate a dataset based on the one or more embeddings in the latent space; and provide a portion of the dataset to a downstream model to cause the downstream model to generate an output, the output of the downstream model based on the portion of the dataset.

In some aspects, the one or more processors can be further configured to obtain the data associated with the set of images from at least one sensor supported by a robotic surgical system.

In some aspects, the one or more processors can be further configured to provide the image of the sampled set of images as input to the model, the model including one or more layers associated with an attention function.

In some aspects, the one or more processors can be further configured to provide the image of the sampled set of images as input to the model, the model trained based on one or more operations performed by the model and a second model.

In some aspects, the one or more operations performed by the model and the second model include augmenting at least one training image associated with a training dataset including images generated during training procedures to generate a first augmented image and a second augmented image. The at least one training procedure can include a medical procedure that is different from the at least one medical procedure.

In some aspects, the one or more operations performed by the model and the second model can include: providing the first augmented image to the model to cause the model to generate a first training output. The one or more operations can include providing the second augmented image to the second model to cause the second model to generate a second training output. The one or more operations can include determining a loss based on a difference between the first training output and the second training output. The one or more operations can include updating weights of the model or the second model based on the loss.

In some aspects, the one or more processors can be further configured to update the data associated with the set of images by sampling the set of images at a predetermined rate.

In some aspects, the one or more processors can be further configured to update the data associated with the set of images by removing metadata identifying at least one individual represented by the set of images.

In some aspects, the one or more processors can be further configured to provide the portion of the dataset to the downstream model, the downstream model implemented by an edge device that is in communication with the one or more processors.

In some aspects, the portion of the dataset can include the at least one embedding corresponding to the set of images. The one or more processors configured to provide the portion of the dataset to the downstream model to cause the downstream model to generate the output can be further configured to train the downstream model based on the portion of the dataset including the at least one embedding.

In some aspects, the one or more processors can be further configured to obtain data associated with at least one second embedding corresponding to at least one second image. The one or more processors can be further configured to generate a few-shot input based on the at least one second embedding and the at least one embedding corresponding to the at least one image, and provide the few-shot input to the downstream model to cause the downstream model to generate the output.

In some aspects the one or more processors can be further configured to obtain data associated with a second embedding corresponding to a second image. The one or more processors can be further configured to update the downstream model based on the data associated with the second embedding. The set of images can be associated with a plurality types of procedures. The second image is associated with a type of procedure from among the plurality of types of procedures.

In some aspects, the one or more processors can be further configured to obtain data associated with a second embedding corresponding to a second image. The one or more processors can be further configured to compare the at least one embedding with the second embedding to determine a difference. The one or more processors can be further configured to compare the difference to a threshold value and determine that the second image is outside of a distribution of images associated with the set of images.

In some aspects, the second image can include a plurality of images. The one or more processors can be further configured to compare a quantity of the plurality of images to a second threshold value and determine that the quantity satisfies the second threshold value. The one or more processors can be further configured to update the model or the downstream model based on the quantity satisfying the second threshold value.

In some aspects, the techniques described herein relate to a method. The method can include obtaining, by one or more processors coupled with memory, one or more frames of a video captured by a camera of a medical procedure performed with a robotic medical system. The method can include generating, by the one or more processors, using a first model trained with self-supervised machine learning, features for the one or more frames. The method can include constructing, by the one or more processors, a dataset based on the generated features. The method can include inputting, by the one or more processors, the dataset into a second model to detect an aspect of the medical procedure.

In some aspects, the techniques described herein relate to a non-transitory computer-readable medium storing processor-executable instructions that, when executed by one or more processors, cause the one or more processors to: obtain one or more frames of a video captured by a camera of a medical procedure performed with a robotic medical system. The instructions can cause the processor to generate, using a first model trained with self-supervised machine learning, features for the one or more frames. The instructions can cause the processor to construct a dataset based on the generated features; and input the dataset into a second model to detect an aspect of the medical procedure.

### BRIEF DESCRIPTION OF THE DRAWINGS

FIG. 1 depicts an example system to extract features to compress images generated during medical procedures, according to some embodiments;

FIG. 2 illustrates a flowchart diagram illustrating an example method for extracting features to compress images generated during medical procedures, according to some embodiments;

FIGS. 3A and 3B illustrates example processing pipelines extracting features to compress images generated during medical procedures, according to some embodiments;

FIG. 4 illustrates a diagram of a medical environment; according to some embodiments;

FIG. 5 illustrates a block diagram depicting an architecture for a computer system that can be employed to implement elements of the systems and methods described and illustrated herein.

### DETAILED DESCRIPTION

Following below are more detailed descriptions of various concepts related to, and implementations of, methods, apparatuses, and systems for extracting features to compress images generated during medical procedures. The various concepts introduced above and discussed in greater detail below can be implemented in any of numerous ways. Although the present disclosure is discussed in the context of a surgical procedure, the present disclosure can be applicable to other medical sessions or environments or activities, as well as non-medical activities where the measurement of objects in the field of view of a robotic system is desired.

As will be understood, over the course of a surgery, robotic surgical systems can allow clinicians to view 2D or 3D representations of a work area (e.g., an area within a body of a patient) and preform maneuvers that involve estimation of the dimensions anatomical structures involved in the operation. But systems involved in processing the data generated by these robotic surgical systems can be affected by multiple challenges. First, training deep learning models (e.g., using attention-based architectures as described herein) based on the frames generated by these systems can use significant amounts of data. For supervised learning, this can include annotating the frames with millions of labels for each feature to be identified, and labels can be especially expensive to obtain in surgical applications, because surgical application may require a higher level of precision and accuracy in labels or annotations relative to non-surgical applications. This can restrict the number of different projects that can be researched, particularly by organizations using computing devices with limited computational resources. And because attention-based deep learning models perform well as a dataset size increases, large datasets can lead to exorbitant model training computation requirements (e.g., hundreds of GPU-days) which, again, can prohibit such training on lesser-resourced devices such as edge devices or client devices.

Apart from the resources used to train attention-based models, frames generated during surgical procedures can include potentially sensitive surgical video that can be restricted from leaving the hospital site where it was recorded due to privacy concerns. And in some cases, the collection of data can be precluded due to state or local law. This can make it technically challenging or difficult for smaller hospitals or organizations including multiple hospitals to process or exchange data with others outside of the organization.

Because some models treat all data as equal, surgical procedures involving specialized instrument maneuvers can result in surgeons implementing different maneuvers using robotic medical systems as described herein than a given model is trained to identify. This can lead to reduced model performance as the training data in the training set does not allow for training of more general models adaptable to larger, generalized domains. This can lead to difficulties when training or updating some models, particularly when dealing with models trained to detect anomalies differently than others were trained by virtue of the different model structures and training datasets, resulting in potentially inconsistent classification of anomalies across models.

The systems, methods, apparatuses, and non-transitory computer-readable media described herein address technical problems associated with certain systems and methods involved in processing images or sets of images (e.g., videos). For example, the techniques implemented by the systems and methods described herein involve extracting features from images or sets of images (e.g., surgical videos) to allow for downstream processing of the images. In some examples, techniques implemented by the systems and methods described involve a first model (e.g., an attention-based model such as a vision transformer (ViT) model) to process the images and generate corresponding embeddings in a latent space. The embeddings can subsequently be processed and stored for downstream analysis (e.g., individually or in association with the corresponding images of each respective embedding).

By training models (e.g., a ViT models), as described, and processing images (e.g., individually or as sets corresponding to surgical videos) using such models, the present disclosure enables multiple downstream processes and use cases. For example, the embeddings output by the ViT model can include compressed representations of each image, which can facilitate or allow for the multiple downstream processes and use cases as described herein. And by processing the images of video streams as described herein during or after medical procedures, systems can sample the images at optimal frame rates to both reduce memory or storage resource consumption (e.g., compress the images) and improve downstream model operation. This can, in turn, improve performance and resource consumption by memory-restricted devices such as edge devices and reduce or eliminate corresponding latencies.

Because features can be extracted using models as described herein, compressed representations of data from larger datasets can be provided to resource-restricted devices and allow for few-shot learning on downstream tasks. This can provide a variety of features, the simplest being faster and more comprehensive anatomy detection, milestone detection, procedure segmentation. Indeed, clinicians can implement at least some of the techniques described herein to train models based on a relatively small dataset that they introduce of custom labels, without requiring a dataset scale that can only be achieved by sharing data. Further, downstream models can be specialized as described herein for specific surgeons and hospitals to improve feature detection. Because features are extracted on a general dataset, the corresponding embeddings can be used to directly model data distributions at each hospital site (and for each individual surgeon). This can help capture instances where surgeons have significantly out of distribution cases or techniques, to trigger a finetuning of the feature extractor to those weights. This can also allows a balance between the base model for generalization, and the finetuned model to adjust to the data requirements of a specific location.

Further, by extracting features at edge devices or client devices as opposed to at a centralized data center, are extracted on edge, potentially sensitive data can be retained locally without the need for off-site processing that often is not under the control of the organization generating the data. This allows significantly increased data privacy. Data privacy can be expanded using differential privacy to make it more difficult to recreate input data when viewing the output from a single hospital, and in some instances data can be transmitted between devices or organizations (or made more broadly available) without the need for SSL/TLS protection.

Benefits can also include improved anomaly detection. For example, because features can be extracted and used to create distributions of data, the same feature extractor can be used to identify outliers (either on a single image or entire case basis). Since these feature are used for all models, anomalies will be less represented by the training data, and therefore all models should have lower confidence in performance. This anomaly detection can be used to detect low-confidence predictions and provide more explainable machine learning models, and can also be used to retrigger training of the feature extractor when significant numbers of anomalies are detected over a short time period, indicating data drift.

Finally, because modern temporal architectures are often attention based (e.g., limited to a certain context window based on data input size, model size, and memory available on the training/inference devices) scaling the input size can be difficult. But by scaling down the size of the representation of each frame by several orders of magnitude from frames to embeddings, the presently-disclosed systems and methods can greatly increase the context window for these temporal attention-based models, allowing training to help models learn long range information about procedures. This can be especially helpful in learning information about surgical workflow, which can have interactions ranging over hours of video.

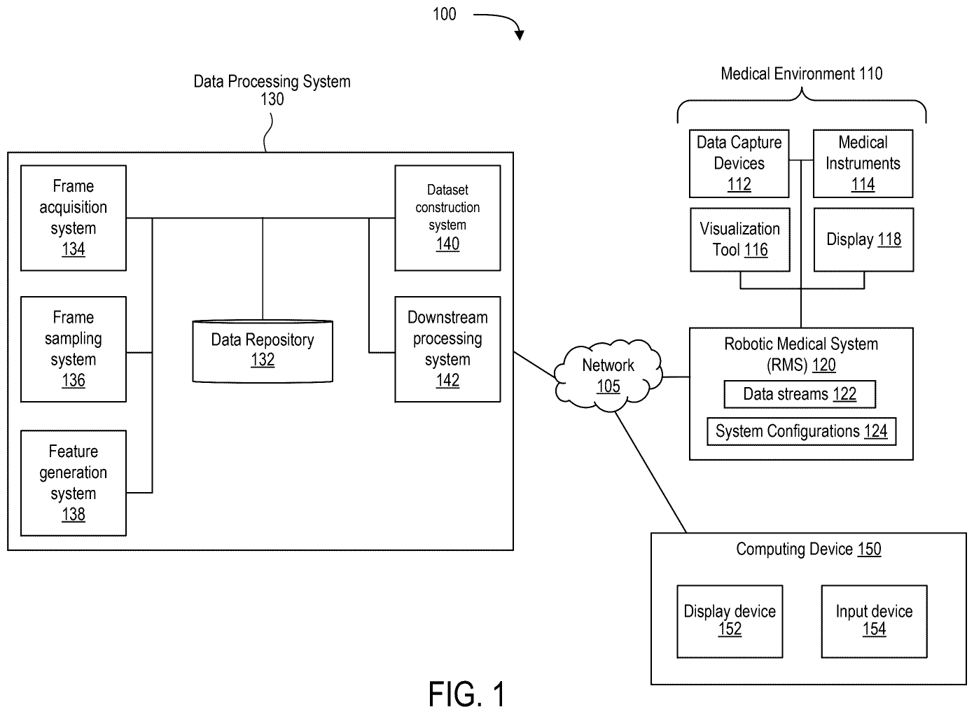

FIG. 1 depicts an example system 100 to extracting features to compress images generated during medical procedures such as, for example, images generated based on use of robotic medical systems during robot-assisted surgeries. The example system 100 can include a combination of hardware and software for generating graphical user interfaces representing aspects of teleoperation of robotic systems. For example, the example system 100 can include a network 105, a medical environment 110, a data processing system 130, and a computing device 150 as described herein.

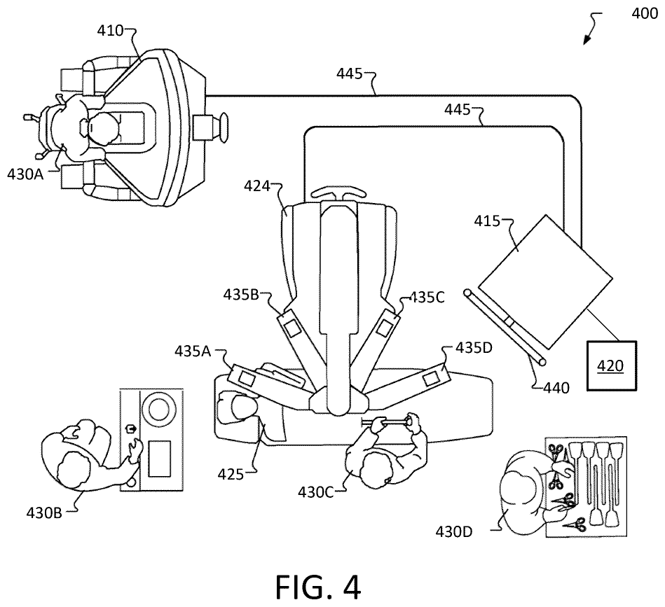

The example system 100 can include a medical environment 110 (e.g., a medical environment that is the same as, or similar to, the example medical environment 400 of FIG. 4) including one or more data capture devices 112, medical instruments 114, visualization tools 116, displays 118 and robotic medical systems (RMSs) 120. RMS 120 can include or generate various types of data streams 122 that are described herein, and can operate using system configurations 124. One or more RMSs 120 can be communicatively coupled with one or more data processing systems 130.

The RMS 120 can be deployed in any medical environment 110. The medical environment 110 can include any space or facility for performing medical procedures, such as a surgical facility or an operating room. The medical environment 110 can include medical instruments 114 (e.g., surgical tools used for specialized tasks) that the RMS 120 can use for performing operational procedures, such as surgical patient procedures, whether invasive, non-invasive, or any in-patient or out-patient procedures. The RMS 120 can be centralized or distributed across a plurality of computing devices or systems, such as computing devices 500 (e.g., used on servers, network devices or cloud computing products) to implement various functionalities of the RMS 120, including communicating or processing data streams 122 across various devices via the network 105.

The medical environment 110 can include one or more data capture devices 112 (e.g., optical devices, such as cameras or sensors or other types of sensors or detectors) for capturing data streams 122. The data streams 122 can include any sensor data, such as images or videos of a surgery, kinematics data on any movement of medical instruments 114, or any events data, such as installation, configuration or selection events corresponding to medical instruments 114. The medical environment 110 can include one or more visualization tools 116 to gather the captured data streams 122 and process it for display to the user (e.g., a surgeon, a medical professional or an engineer or a technician configuring RMS) via one or more displays 118 (e.g., a touchscreen, an LCD display). A display 118 can present data stream 122 (e.g., images or video frames) of a medical procedure (e.g., surgery) being performed using the RMS 120 while handling, manipulating, holding or otherwise utilizing medical instruments 114 to perform surgical tasks at the surgical site. RMS 120 can include system configurations 124 based at least on which RMS 120 can operate, and the functionality of which can impact the data flow of the data streams 122. As will be described herein, the data streams 122 can be divided into multiple data streams.

The system 100 can include one or more data capture devices 112 (e.g., video cameras, sensors or detectors) for collecting data streams 122, that can be used for machine learning, including detection of objects from sensor data (e.g., video frames or force or feedback data), detection of particular events (e.g., user interface selection of, or a surgeon's engaging of, a medical instrument 114) or detection of kinematics (e.g., movements of the medical instrument 114). The data capture devices 112 can include cameras or other image capture devices for capturing videos or images from a particular viewpoint within the medical environment 110. The data capture devices 112 can be positioned, mounted, or otherwise located to capture content from any viewpoint that facilitates the data processing system capturing various surgical tasks or actions.

The data capture devices 112 can include any of a variety of detectors, sensors, cameras, video imaging devices, infrared imaging devices, visible light imaging devices, intensity imaging devices (e.g., black, color, grayscale imaging devices, etc.), hyperspectral imaging devices (e.g., a hyperspectral camera, etc.), depth imaging devices (e.g., stereoscopic imaging devices, time-of-flight imaging devices, etc.), medical imaging devices such as endoscopic imaging devices, ultrasound imaging devices, etc., non-visible light imaging devices, any combination or sub-combination of the above mentioned imaging devices, or any other type of imaging devices that can be suitable for the purposes described herein. The data capture devices 112 can include cameras that a surgeon can use to perform a surgery and observe manipulation components within a purview of field of view suitable for the given task performance. The data capture devices can output any type of data streams 122, including data streams 122 of kinematics data (e.g., kinematics data streams), data streams 122 of events data (e.g., events data streams) and data streams 122 of sensor data (e.g., sensors data streams).

For example, data capture devices 112 can capture, detect, or acquire sensor data such as videos or images, including for example, still images, video images, vector images, bitmap images, other types of images (e.g., Raman hyperspectral images, etc.), or combinations thereof. The data capture devices 112 can capture the images at any suitable predetermined capture rate or frequency. Settings, such as zoom settings or resolution, of each of the data capture devices 112 can vary as desired to capture suitable images from any viewpoint. For instance, data capture devices 112 can have fixed viewpoints, locations, positions, or orientations. The data capture devices 112 can be portable, or otherwise configured to change orientation or telescope in various directions. The data capture devices 112 can be part of a multi-sensor architecture including multiple sensors, with each sensor being configured to detect, measure, or otherwise capture a particular parameter (e.g., sound, images, or pressure).

The data capture devices 112 can generate sensor data from any type and form of a sensor, such as a positioning sensor, a biometric sensor, a velocity sensor, an acceleration sensor, a vibration sensor, a motion sensor, a pressure sensor, a light sensor, a distance sensor, a current sensor, a focus sensor, a temperature or pressure sensor or any other type and form of sensor used for providing data on the medical instruments 114, or the data capture devices (e.g., optical devices). For example, the data capture device 112 can include a location sensor, a distance sensor or a positioning sensor providing coordinate locations of a medical instrument 114 (e.g., kinematics data). The data capture device 112 can include a sensor providing information or data on a location, position or spatial orientation of an object (e.g., medical instrument 114 or a lens of data capture device 112) with respect to a reference point for kinematics data. The reference point can include any fixed, defined location used as the starting point for measuring distances and positions in a specific direction, serving as the origin from which all other points or locations can be determined.

The display 118 can show, illustrate or play the data stream 122, such as a video stream, in which the medical instruments 114 at or near surgical sites are shown. For example, the display 118 can display a rectangular image of a surgical site along with at least a portion of the medical instruments 114 being used to perform surgical tasks. The display 118 can provide compiled or composite images generated by the visualization tool 116 from a plurality of data capture devices 112 to provide a visual feedback from one or more points of view.

The visualization tool 116 can be configured or designed to receive any number of different data streams 122 from any number of data capture devices 112 and combine them into one or more data streams displayed on a display 118. The visualization tool 116 can be configured to receive a plurality of data stream components and combine the plurality of data stream components into a single data stream 122. For instance, the visualization tool 116 can receive visual sensor data from one or more of the medical instruments 114, sensors or cameras with respect to a surgical site or an area in which a surgery is performed. The visualization tool 116 can incorporate, combine or utilize multiple types of data (e.g., positioning data of a medical instrument 114 along sensor readings of pressure, temperature, vibration or any other data) to generate an output to present on a display 118. The visualization tool 116 can present locations of medical instruments 114 along with locations of any reference points or surgical sites, including locations of anatomical parts of the patient (e.g., organs, glands or bones).

The medical instruments 114 can be any type and form of tool or medical instrument used for surgery, medical procedures or a tool in an operating room or environment. The medical instrument 114 can be imaged by, associated with, or include an image capture device. For instance, a medical instrument 114 can be a tool for making incisions, a tool for suturing a wound, an endoscope for visualizing organs or tissues, an imaging device, a needle and a thread for stitching a wound, a surgical scalpel, forceps, scissors, retractors, graspers, or any other tool or medical instrument to be used during a surgery. The medical instruments 114 can include hemostats, trocars, surgical drills, suction devices or any medical instruments for use during a surgery. The medical instrument 114 can include other or additional types of therapeutic or diagnostic medical imaging implements. The medical instrument 114 can be configured to be installed in, coupled with, or manipulated by an RMS 120, such as by manipulator arms or other components for holding, using and manipulating the medical instruments. The medical instruments 114 can be the same as, or similar to, the medical instruments discussed with respect to FIG. 4.

The RMS 120 can be a computer-assisted system configured to perform a surgical or medical procedure or activity on a patient via, or using or with the assistance of, one or more robotic components or the medical instruments 114. The RMS 120 can include any number of manipulator arms for grasping, holding or manipulating various medical instruments 114 and performing computer-assisted medical tasks using the medical instruments 114 controlled by the manipulator arms.

The data streams 122 can be generated by the RMS 120. For instance, sensor data associated with the data streams 122 can include images (e.g., video images) captured by a medical instrument 114 and can be sent to the visualization tool 116. For instance, a display 118 (e.g., a touchscreen) can be used by a surgeon to select, engage, or configure a particular medical instrument 114, thereby triggering an event that can be indicated or included in data packets of a data stream 122. The RMS 120 can include one or more input ports to receive direct or indirect connection of one or more auxiliary devices. For example, the visualization tool 116 can be connected to the RMS 120 to receive the images from the medical instrument 114 when the medical instrument 114 is installed in the RMS 120 (e.g., on a manipulator arm for handing medical instruments 114). For example, the data stream 122 can include data indicative of positioning and movement of the medical instruments 114 that can be captured or identified by data packets of a kinematics data. The visualization tool 116 can combine the data stream components from the data capture devices 112 and the medical instrument 114 into a single combined data stream 122 which can be indicated or presented on a display 118. The RMS 120 can provide the data streams 122 to the data processing system 130 periodically, continuously, or in real-time.

Data packets can include a unit of data in a data stream 122. The data packets can include the actual information being sent and metadata, such as a source and a destination address, a port identifier or any other information for transmitting data. The data packets can include a data (e.g., a payload) corresponding to an event (e.g., installation, uninstallation, engagement or setup of a medical instrument 114). The data packets can include data corresponding to sensor information (e.g., a video frame captured by a camera), or data on movement of a medical instrument 114. The data packets can be transmitted in the data streams 122 that can be separated or combined. For instance, a data stream 122 for kinematics data (e.g., a kinematics data stream) can include a plurality of data packets indicative of movement of robotic system components or features.

Data packets can include one or more timestamps, which can indicate a particular time when particular events took place. Timestamps can include time indications expressed in any combination of nanoseconds, microseconds, milliseconds, seconds, hours, days, months or years. Timestamps can be included in the payload or metadata of data packets and can indicate the time when a data packet was generated, the time when the data packet was transmitted from the device that generated the data packet, the time when the data packet was received by another device (e.g., a system within the RMS 120, the data processing system 130, the computing device 150 or another device on a network) or a time when the data packet is stored into a data repository 132.

The data repository 132 can include one or more data files, data structures, arrays, values, or other information that facilitates operation of the data processing system 130. The data repository 132 can include one or more local or distributed databases and can include a database management system. The data repository 132 can include, maintain, or manage one or more data streams 122. The data streams 122 can include or be formed from one or more of a video stream, image stream, stream of sensor measurements, event stream, or kinematics stream. The data streams 122 can include data collected by one or more data capture devices 112, such as a set of 3D sensors from a variety of angles or vantage points with respect to the procedure activity (e.g., point or area of surgery).

The data stream 122 can include any stream of data. The data stream 122 can include a video stream, including a series of video frames or organized into video fragments, such as video fragments of about 1, 2, 3, 4, 5, 10 or 15 seconds of a video. Each second of the video can include, for example, 30, 45, 60, 90, 120, 240 video frames per second. The data streams 122 can include an event stream which can include a stream of event data or information, such as packets, which identify or convey a state of the RMS 120 or an event that occurred in association with the RMS 120. For example, data stream 122 can include any portion of system configuration 124, including information on operations on data streams 122, data on installation, uninstallation, calibration, set up, attachment, detachment or any other action performed by or on an RMS 120 with respect to the medical instruments 114.

The data stream 122 can include data about an event, such as a state of the RMS 120 indicating whether the medical instrument 114 is calibrated, adjusted or includes a manipulator arm installed on the RMS 120. A data stream 122 representing event data (e.g., event data stream) can include data on whether an RMS 120 was fully functional (e.g., without errors) during the procedure. For example, when a medical instrument 114 is installed on a manipulator arm of the RMS 120, a signal or data packet(s) can be generated indicating that the medical instrument 114 has been installed on the manipulator arm of the RMS 120.

The data stream 122 can include a stream of kinematics data which can refer to or include data associated with one or more of the manipulator arms or medical instruments 114 attached to the manipulator arms, such as arm locations or positioning. The data corresponding to the medical instruments 114 can be captured or detected by one or more displacement transducers, orientational sensors, positional sensors, or other types of sensors and devices to measure parameters or generate kinematics information. The kinematics data can include sensor data along with time stamps and an indication of the medical instrument 114 or type of medical instrument 114 associated with the data stream 122.

The data repository 132 can store sensor data having video frames that can include one or more static images or frames extracted from a sequence of images of a video file. Video frame can represent a specific moment in time and can be identified by a metadata including a timestamp. Video frames can display visual content of the video of a medical procedure being analyzed by the data processing system 130 to form a composite video along with performance metrics indicative of the performance of the surgeon performing the procedure. For example, in a video file capturing a robotic surgical procedure, a video frame can depict a snapshot of the surgical task, illustrating a movement or usage of a medical instrument 114 such as a robotic arm manipulating a surgical tool within the patient's body.

The data streams 122 corresponding to sensor data (e.g., videos), events, and kinematics can include related, corresponding or duplicate information that can be used for cross-data comparisons and verification that all three data sources are in agreement. For instance, the detection function can implement a check for consistency between diverse data types and data sources by mapping and comparing timestamps between different data types to facilitate if they consistently progress over time, such as in accordance with expected flow and correlation of events, video stream details and kinematics values.

For example, an installation of a medical instrument 114 can be recorded as a system event and provided in a data stream 122 of events data. At the same or similar expected time frame, the installed medical instrument 114 can shows up in a sensor data (e.g., in a video) which can be detected by the data processing system 130, which can include a computer vision model. Kinematics data can confirm movements of the medical instrument 114 according to the movements detected by the data processing system 130. Using these cross-data stream correlation techniques, the data processing system 130 can verify time synchronization across the three data sources (e.g., three data streams 122).

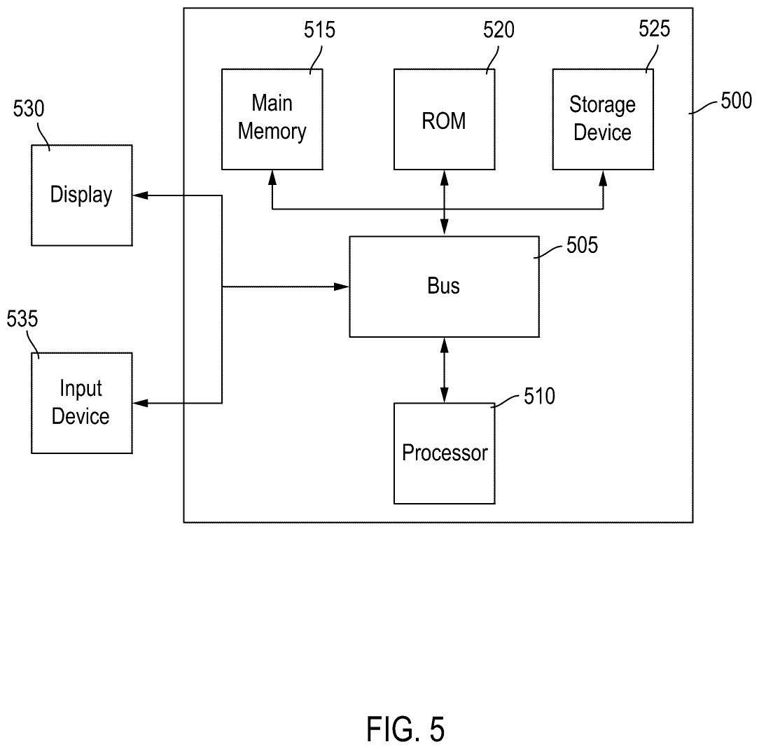

With continued reference to FIG. 1, among others, the data processing system 130 can include any combination of hardware or software that performs one or more of the functions described herein. For example, the data processing system 130 can include any combination of hardware and software for extracting features to compress images generated during medical procedures. The data processing system 130 can include any computing device (e.g., a computing device that is the same as, or similar to, the computing device 500 of FIG. 5) and can include one or more servers, virtual machines, or can be part of or include a cloud computing environment. The data processing system 130 can be provided via a centralized computing device or be provided via distributed computing components, such as including multiple, logically grouped servers and facilitating distributed computing techniques. The logical group of servers can be referred to as a data center, server farm or a machine farm. The servers, which can include virtual machines, can also be geographically dispersed. A data center or machine farm can be administered as a single entity, or the machine farm can include a plurality of machine farms. The servers within each machine farm can be heterogeneous-one or more of the servers or machines can operate according to one or more type of operating system platform.

The data processing system 130, or components thereof can include a physical or virtual computer system operatively coupled, or associated with, the medical environment 110. The data processing system 130, or components thereof, can be coupled, or associated with, the medical environment 110 via a network 105, either directly or indirectly through an intermediate computing device or system. The network 105 can be any type or form of network. The geographical scope of the network can vary widely and can include a body area network (BAN), a personal area network (PAN), a local-area network (LAN) (e.g., Intranet), a metropolitan area network (MAN), a wide area network (WAN), or the Internet. The topology of the network 105 can assume any form such as point-to-point, bus, star, ring, mesh, tree, etc. The network 105 can utilize different techniques and layers or stacks of protocols, including, for example, the Ethernet protocol, the internet protocol suite (TCP/IP), the ATM (Asynchronous Transfer Mode) technique, the SONET (Synchronous Optical Networking) protocol, the SDH (Synchronous Digital Hierarchy) protocol, etc. The TCP/IP internet protocol suite can include application layer, transport layer, internet layer (including, e.g., IPv6), or the link layer. The network 105 can be a type of a broadcast network, a telecommunications network, a data communication network, a computer network, a Bluetooth network, or other types of wired and wireless networks.

The data processing system 130, or components thereof, can be located at least partially at the location of the surgical facility associated with the medical environment 110 or remotely therefrom. Elements of the data processing system 130, or components thereof can be accessible via portable devices such as laptops, mobile devices, wearable smart devices, etc., including the computing device 150. The data processing system 130, or components thereof, can include other or additional elements that can be considered desirable to have in performing the functions described herein. The data processing system 130, or components thereof, can include, or be associated with, one or more components or functionality of a computing device including, for example, one or more processors coupled with memory that can store instructions, data or commands for implementing the functionalities of the data processing system 130 discussed herein.

The data processing system 130 can include one or more of a data repository 132 configured to store one or more datasets, a frame acquisition system 134, a frame sampling system 136, a feature generation system 138, a dataset construction system 140, or a downstream processing system 142. While each of the systems of the data processing system 130 are described as being configured to perform one or more operations, the components can cooperate with one or more different components of the data processing system 130 to perform the one or more described operations. The data processing system 130 can be communicatively coupled with one or more data processing systems (not explicitly illustrated) that operate in cooperation to perform one or more of the operations described herein.

The data processing system 130 can be implemented by one or more components of the medical environment 110. For example, the data processing system 130 can be implemented by one or more components of the RMS 120. The data processing system 130 can receive one or more data streams 122 that are described herein, and can monitor operation of the RMS 120 using the system configurations 124. One or more RMSs 120 can be communicatively coupled with the one or more data processing systems 130. The data repository 132 can be configured to receive, store, and provide the data streams 122 (e.g., one or more data packets associated with the data streams 122) before, during, or after a medical procedure to one or more other devices of FIG. 1.

The data repository 132 can be implemented by the data processing system 130 or can be a device that is the same as, or similar to, the computing device 500 of FIG. 5. The data repository 132 can receive data from, or transmit data to, any of the devices of FIG. 1, either directly or indirectly (e.g., via the data processing system 130). The data can include the data streams 122. In examples, the data stored by the data repository 132 is associated with a currently- or previously-performed medical procedure involving the RMS 120 or another robotic system. The data repository 132 can receive the data streams 122 or the system configurations 124 and store the data streams 122 or the system configurations 124 therein. The data repository 132 can provide the data streams 122 or the system configurations 124 (e.g., one or more data packets thereof) to the one or more of the components of the data processing system 130. The data repository 132 stores data associated with one or more of data streams 122 generated during operation of at least one component of the RMS 120 or data generated by the components of the data processing system 130.

The frame acquisition system 134 can be implemented by the data processing system 130 or can be a device that is the same as, or similar to, the computing device 500 of FIG. 5. The frame acquisition system 134 can obtain (e.g., receive) the data streams 122. For example, the frame acquisition system 134 can receive the data streams 122 via the network 105. In examples, the frame acquisition system 134 can receive the data streams 122 via the data repository 132. The frame acquisition system 134 can receive the data streams 122 of a medical procedure performed with the RMS 120. For example, the frame acquisition system 134 can receive the data streams 122, where the data streams 122 include data (e.g., packets) generated by one or more devices of the RMS 120 or in communication with the RMS 120 during the medical procedure. The one or more packets associated with the data streams 122 can include (e.g., represent) one or more images (e.g., single images or sets of images representing a video stream) generated by at least one camera during a medical procedure or one or more movements of the medical instruments 114 or MTMs of the RMS 120 engaged by an individual during the medical procedure to at least in part cause movement of the medical instruments 114. The MTMs can include one or more devices configured to be grasped by the individual and moved within an area along six degrees of freedom and the movements can be tracked by the RMS 120 to at least in part cause the medical instruments 114 to move in accordance with the movements of the MTMs.

The one or more images of the medical procedure can be captured as frames or otherwise obtained by the data capture devices 112 or the visualization tool 116. The images included in the data streams 122 can be generated by an imaging device that is supported by a distal portion of a medical instrument 114 (e.g., an endoscope). The one or more images can represent one or more anatomical structures (or portions thereof) or portions of the one or more medical instruments 114.

The frame sampling system 136 can obtain one of more frames (e.g., representing one or more images) from the frame acquisition system 134. For example, the frame sampling system 136 can obtain the one or more frames from the frame acquisition system 134 as frames are generated during operation of the RMS 120. In examples, the frame sampling system 136 can obtain the one or more frames from the frame acquisition system 134 based on the data processing system 130 receiving a request to process the one or more frames.

The frame sampling system 136 can sample (e.g., select) one or more frames from among a plurality of frames. For example, the frame sampling system 136 can select one or more frames from among a plurality of frames corresponding to a surgical procedure at a predetermined rate (e.g., 1 frame per second (fps), 2 fps, or 3 fps). The frame sampling system 136 can select the one or more frames based on a predetermined rate corresponding to one or more of a type of procedure represented by the frames, a phase of the procedure represented by the frames, a complexity of the procedure, a complexity of a phase of the procedure, a technique implemented during a surgical procedure, a relative size of the anatomical feature being manipulated during at least a portion of a surgical procedure, whether a technique was implemented manually or not manually (e.g., hand sewn vs. stapled), or combinations thereof. For example, the frame sampling system 136 can select the one or more frames based on a frame sample rate corresponding to the type of procedure represented by the frames, the phase of the procedure represented by the frames, the complexity of the procedure, the complexity of the phase of the procedure. In an example, frame rates for types of procedures are included below in Table 1; frame rates for phases of procedures are included in Table 2; frame rates for complexities of procedures in Table 3; frame rates for complexity of procedures at given phases in Table 4; frame rates based on a relative size of an anatomical structure being manipulated during a surgical procedure in Table 5; and frame rates based on techniques implemented during a surgical procedure in Table 6.

TABLE 1
Frame rates based on types of procedures

| Type of procedure | Frame rate |
|---|---|
| Hysterectomy | 5 fps |
| Cholecystectomy | 2 fps |
| Tracheal resection | 10 fps |
| Lung tumor removal | 2 fps |

TABLE 2
Frame rates based on phases of procedures

| Phase of a procedure | Frame rate | |
|---|---|---|
| Specimen removal | 0.1 | fps |
| Skeletonization and division of an artery/vein | 10 | fps |
| Removal of adhesions | 1 | fps |

TABLE 3
Frame rates based on overall procedure complexity

| Type of procedure | Frame rate |
|---|---|
| Low complexity | 2 fps |
| Medium complexity | 10 fps |
| High complexity | 20 fps |

TABLE 4
Frame rates based on phase complexity

| Type of procedure | Frame rate |
|---|---|
| Incision phase (low complexity) | 1 fps |
| Navigation phase (medium complexity) | 5 fps |
| Endoscopic mucosal resection phase (high complexity) | 10 fps |
| Instrument removal phase (low complexity) | 1 fps |
| Suturing phase (low complexity) | 1 fps |

TABLE 5
Frame rates based on a relative size of an anatomical
structure being manipulated during a surgical procedure

| Type of procedure | Frame rate |
|---|---|
| Small bowel | 1 fps |
| Cystic duct | 10 fps |
| Gastric fundus | 1 fps |
| Crura of the esophagus | 5 fps |

TABLE 6
Frame rates based on how techniques are
implemented during a surgical procedure

| Type of procedure | Frame rate |
|---|---|
| Hand sewn | 5 fps |
| Stapled | 2 fps |

In this way, the frame sampling system 136 can set the rate at which the frames are sampled so as to sample more complex surgical procedures or surgical procedures that involve quicker movements of the instruments involved at higher rates that surgical procedures that are less complex (e.g., require less maneuvers) or involve slower movements. For example, the frame sampling system 136 can set the rate at which the frames are sampled such that less predictable and repeatable techniques (e.g., hand sewn/manual anastomoses) are sampled at higher rates when compared to more predictable and repeatable techniques (e.g., stapling anastomoses) to increase the granularity of information available to measure the skill of a clinician performing these steps. In examples, the frame sample system 136 can set the rate at which the frames are sampled such that techniques where surgeons dissect tissue involving small or delicate structures (e.g., where additional care or attention can be beneficial) are sampled at higher rates to increase the granularity of information available to represent these shorter, finer tasks when compared to dissection of tissue involving larger or less delicate structures. In some examples, the frame sample system 136 can set the rate at which the frames are sampled such that techniques where surgeons implement techniques of greater clinical significance (e.g., skeletonization of an artery) are sampled at higher rates when compared to techniques of lesser clinical significance (e.g., specimen removal where metrics are focused on determining whether the step was performed or not performed and less the manner in which the step was performed).

The frame sample system 136 can sample the one or more frames based on input provided by a clinician. For example, a clinician can provide input (e.g., via the input device 154 of the computing device 150) including a request to detect a type of aspect of a medical procedure. In examples, aspects can include a phase of a medical procedure, a technique capable of being performed of the medical procedure, a type of the medical procedure, etc. The input can then be provided to the data processing system 130, which can communicate the input to the frame sampling system 136. In this example, the frame sampling system 136 can select the frame rate based on the type of aspect.

The frame sampling system 136 can update one or more frames (e.g., the one or more images of the one or more frames) of from among the plurality of frames. For example, the frame sampling system 136 can update the one or more frames to generate a sampled set of frames. In this example, the sampled set of frames can be updated based on the frame sampling system 136 selecting a predetermined rate at which the frames are sampled. The sampled set of frames can be updated to remove metadata identifying the individual involved in the procedure (e.g., the patient). For example, the frame sampling system 136 can remove the metadata for each frame that is sampled. The frame sampling system 136 can provide the frames that were sampled (e.g., the sampled set of frames) as input to the feature generation system 138.

The feature generation system 138 can generate one or more embeddings based on one or more frames processed by the data processing system 130. For example, the feature generation system 138 can generate the one or more embeddings based on the sampled set of images. The feature generation system 138 can generate the one or more embeddings by providing each frame of the sampled set of images to a first model (e.g., a machine learning model that is configured to perform one or more attention-based operations such as a vision transformer (ViT)). For example, the feature generation system 138 can generate the one or more embeddings by providing each frame to the first model to cause the first model to generate an output. In this example, the output can include an embedding. The embedding can be a high-dimensional vector representation of the frame (or a part of the frame). In examples, the embedding can capture the visual features and semantic information of the frame, enabling the first model or other models to perform operations involved in, for example, image classification, object detection, image generation. In examples described herein, the embedding can capture visual features or semantic information that correspond to aspects of medical procedures.

The feature generation system 138 can generate the one or more embeddings based on the one or more frames processed by the data processing system 130 or one or more aspects associated with the one or more frames. For example, the feature generation system 138 can generate the one or more embeddings based on multimodal data, kinematics data, event stream data, or combinations thereof, where the multimodal data, kinematics data, event stream data corresponds to the one or more frames. The multimodal data can be generated by one or more of the devices of the medical environment 110 and represented by the data generated by the RMS 120 (e.g., the data streams 122 or the system configurations 124). In examples, the multimodal data can include data associated the frames or text generated based on the frames. The text can be generated based on input provided by clinicians at the input device 154 of the computing device 150 or based on the RMS 120 annotating the frames based on generation of the frames during corresponding surgical procedures. In examples, the RMS 120 can annotate the frames to include text indicating one or more aspects of a surgical procedure such as a type of the surgical procedure, a phase of the surgical procedure, or one or more instruments used during the surgical procedure at one or more points in time. The kinematics data can be generated based on operation of the medical instruments 114. For example, a robotic medical system as described herein supporting one or more of the medical instruments 114 can generate the kinematics data based on changes in position of the medical instruments 114 or one or more components thereof. The event stream data can include a stream of event data or information generated by the RMS 120 during a surgical procedure. For example, the even stream data can include packets which identify or convey a state of the RMS 120, or an event that occurred in association with the RMS 120 during a surgical procedure.

The multimodal data, the kinematics data, the event stream data, or combinations thereof, can be provided to the data processing system 130 with the data associated with the one or more frames. For example, the RMS 120 can generate the multimodal data, the kinematics data, or the event stream data and include the data (or combinations thereof) with corresponding data associated with the frames. In this example, the multimodal data, the kinematics data, or the event stream data can be included as separate channels and provided to one or more models as described herein.

The feature generation system 138 can provide frames or data associated with the channels for the frames (e.g., the kinematics data, the event stream data, or combinations thereof) to the first model to cause the first model to generate the embeddings in a latent space. For example, the feature generation system 138 can provide frames associated with one or more medical procedures or data associated with the channels for the frames to one or more layers of the first model to cause the layers to cooperatively perform operations to generate the embeddings as an output. In this example, one or more of the layers can be associated with attention functions that implement multi-head self-attention. In this example, the layers can take as input a set of queries, keys, and values associated with one or more frames, and compute a weighted sum of the values based on the similarity between the queries and keys. The attention weights can be computed using a dot product of the queries and keys, followed by a softmax function to normalize the weights. In the context of attention-based models such as vision transformers, the queries, keys, and values can be obtained from patches of the frames or data associated with the channels for the frames to allow the first model to learn to focus on relationships between different parts of the image based on the context and the task at hand.

The feature generation system 138 can select the first model based on a type of aspect of a medical procedure. For example, the feature generation system 138 can select the first model from among a plurality of models as described herein that are trained on different frames representing different features. The feature generation system 138 can select the model based on input provided by a clinician. For example, a clinician can provide input (e.g., via the input device 154 of the computing device 150) including a request to detect a type of aspect of a medical procedure (e.g., an anatomical structure of a medical procedure, a milestone of a medical procedure (e.g., successful execution of one or more phases of a medical procedure such as stapling, cauterization of a blood vessel, ablation, or ligation), a phase of a medical procedure (e.g., an incision phase, a navigation phase, an endoscopic mucosal resection phase, an instrument removal phase, or a suturing phase), a task of a medical procedure (e.g., insufflation). The input can then be provided to the data processing system 130, which can communicate the input to the feature generation system 138. In this example, the feature generation system 138 can select the first model based on the type of aspect.

In examples, subsets of the frames or data associated with the channels for the frames can be associated with medical procedures having particular types, phases of medical procedures. In this example, where the first model is an attention-based model such as a vision transformer, the latent space for the embeddings output by the vision transformer can be associated with a learned, high-dimensional vector space where each dimension represents a latent feature (e.g., anatomical features involved in procedures, pathological features involved in the procedures, spatial relationships between anatomical structures or pathological features, temporal changes (e.g., from frame to frame)) associated with a domain (e.g., surgical procedures). The embeddings generated by the vision transformer can be projected into the latent space, where similar frames or patches of the frames are mapped to similar embeddings within the latent space, and dissimilar frames or patches of the frames are mapped to distant embeddings. As will be understood, this latent space can be learned during training of the attention-based model by the components of the data processing system 130. This can allow the attention-based model to capture meaningful relationships between different frames or areas within a given frame or set of frames.

The feature generation system 138 can generate the first model based on operations performed by the first model and a second model. For example, the feature generation system 138 can initialize the first model and a second model, where the first model and the second model are both attention-based models (e.g., vision transformers that can be the same as one another). At least a portion of a frame from a training set of frames can be provided to both models. For example, for a given frame, the first model can receive a first patch of the frame that is less than half the size of the frame and a second patch of the frame that is greater than half the size of the frame. The second model can also receive the second patch of the frame. The first model and the second model can then process their respective inputs and generate a first training output (e.g., a first embedding) and a second training output (e.g., a second embedding) respectively. The first training output and the second training output can then be compared to determine a loss (e.g., a cross-entropy loss) between the two. The weights of the first model can be updated based on the loss (also referred to as backpropagation), and the weights of the second model can be updated based on an exponential moving average that is determined based on the weights of the first model. In this way, the first model can be trained based on the implementation of self-knowledge distillation techniques or self-supervision to learn representations within the frames, allowing the first model to encode features to contain long-range spatial representations.

When generating the first model, the feature generation system 138 can augment the patches before providing the patches as input to the models such that the first patch of the frame represents a first augmented patch (e.g., a first augmented image) and the second patch of the frame represents a second augmented patch (e.g., a second augmented image). The feature generation system 138 can obtain frames and augment patches of the frames as described here, where the frames are associated with a domain that is different from a domain that for which the first model is being trained to generate embeddings. For example, the feature generation system 138 can obtain frames of medical procedures involving surgeries to address issues with an abdomen of a patient (referred to as first domain frames) and generate the patches of the frames to train the first model based on the first domain frames. In this example, the feature generation system 138 can obtain frames of a medical procedure involving surgeries to address issues with a spine of a patient (referred to as second domain frames) and generate the patches of the frames to train the first model based on the second domain frames to allow the first model to obtain subsequent frames and generate embeddings for the frames as described herein.

The feature generation system 138 can provide data associated with the first model to the dataset construction system 138. For example, the feature generation system 138 can obtain and process a plurality of frames or data associated with the channels for the frames associated with a type of medical procedure using the first model to generate corresponding embeddings for each frame. The feature generation system 138 can then provide the frames or embeddings to the dataset construction system 140 to allow the dataset construction system 140 to generate a dataset as described herein. In examples, the feature generation system 138 can obtain and process a first plurality of frames associated with a first type of medical procedure and a second plurality of frames associated with a second type of medical procedure. In these examples, the feature generation system 138 can process the first plurality of frames or data associated with the channels for the frames associated with the first type of medical procedure to generate a first set of embeddings and process the second plurality of frames or data associated with the channels for the frames associated with the second type of medical procedure to generate the second set of embeddings. In these examples, the feature generation system 138 can provide the data associated with embeddings to the dataset construction system 140.

The dataset construction system 140 can generate a dataset based on one or more embeddings associated with one or more medical procedures. For example, the dataset construction system 140 can generate a dataset based on one or more embeddings associated with one or more latent spaces. When generating a dataset for a given laten space, the dataset construction system 140 can obtain embeddings from the feature generation system 138 and compare the embeddings to each other to determine differences between the embeddings. In one example, where the dataset construction system 140 obtains a first embedding and a second embedding that are associated with the same medical procedure, the same phase of the medical procedure, the dataset construction system can compare the first embedding and the second embedding to determine a difference between the embeddings. The dataset construction system can then compare the difference to a threshold value associated with embeddings in a latent space. Where the difference satisfies the threshold value (e.g., indicating that the first embedding and the second embedding are in the same latent space), the dataset construction system 140 can include the one or more embeddings in the dataset. Where the difference does not satisfy the threshold value (e.g., indicating that the first embedding and the second embedding are not in the same latent space), the dataset construction system 140 can forgo including the one or more embeddings in the dataset. the dataset construction system 140 can then provide the dataset including the embeddings to the downstream processing system 142.

The downstream processing system 142 can provide a portion of the dataset to a downstream model (e.g., a second model) to cause the downstream model to generate an output. For example, the downstream processing system 142 can provide the portion of the dataset to downstream model to cause the downstream model to generate an output based on the embeddings included in the portion of the dataset. In the examples described, the downstream model can be implemented by the data processing system 130 or by a an edge device (e.g., the computing device 150) that is in communication with the data processing system 130.

The downstream processing system 142 can provide the portion of the dataset to the downstream model to cause the computing device implementing the downstream model to train the downstream model. For example, the downstream processing system 142 can provide the portion of the dataset to the downstream model to train the downstream model using the at least one embedding. In this example, the downstream model can include another attention-based model (e.g., another vision transformer), or other types of neural networks (e.g., convolutional neural networks (CNNs), autoencoders, U-nets). The downstream processing system 142 can select the downstream model based on an attribute associated with the first model. The attribute can include, for example, a type of medical procedure or a type of feature for one or more medical procedures for which the first model is configured to generate embeddings.

The downstream processing system 142 can select the second model based on a type of aspect of a medical procedure. For example, the downstream processing system 142 can select the second model from among a plurality of models that are trained on different frames representing different features. In some examples, the downstream processing system 142 can select the second model where a goal of the second model is to segment one or more portions of the frames. In these examples, the downstream processing system 142 can select a U-net or ViT as the second model and train the second model based on the task of segmenting objects (e.g., anatomical features represented by the embeddings). In another example, the downstream processing system 142 can select a convolutional neural network (CNN) as the second model and train the second model based on the task of classifying objects to indicate, for example, a type of anatomical feature present in one or more frames of one or more videos. In this example, the selection and training of a CNN can allow for analysis and classification of more complex features with each successive convolutional layer.

The downstream processing system 142 can select the second model based on input provided by a clinician. For example, a clinician can provide input (e.g., via the input device 154 of the computing device 150) including a request to detect a type of aspect of a medical procedure. The input can then be provided to the data processing system 130, which can communicate the input to the downstream processing system 142. In this example, the downstream processing system 142 can select the second model based on the type of aspect.

The downstream processing system 142 can select the downstream model based on a characteristic of a type of medical procedure to be analyzed. For example, the downstream processing system 142 can select the downstream model based on a type of medical procedure being researched. In this example, the downstream system 142 can select a dataset generated by a dataset construction system 140 based on a first model configured to generate embeddings relevant to the research. The downstream processing system 142 can then select the second model (e.g., where the second model includes a CNN, an autoencoder) based on a characteristic of the target procedure being researched (e.g., one or more anatomic features being operated on). In this way, the data processing system 130 can select, train, or update a first model and a downstream model to optimize the detection of targeted characteristics during a medical procedure.

In some examples, the downstream processing system 142 can generate a few-shot input to train the downstream model. For example, the downstream processing system 142 can provide one or more embeddings of the dataset and input data (e.g., a frame or an embedding that is not from the dataset) to cause the downstream model to generate an output. In this way, the downstream processing system 142 can include one or more embeddings in the known latent space (e.g., associated with one or more predetermined features of a given medical procedure or phase of a medical procedure) with the input data to enable the downstream model to generalize from the examples and generate an output for a specific medical procedure that can include or not include the medical procedure associated with the dataset.

The downstream processing system 142 can generate a training dataset to be used when training the downstream model. For example, the downstream processing system 142 can obtain a dataset comprising embeddings involved in a plurality of types of procedures and provide the dataset to generate or update the downstream model to generate outputs. The downstream processing system 142 can compare a quantity of embeddings corresponding to frames of a given type of medical procedure to a threshold value (e.g., indicating a minimum number of frames to use when training downstream models for given medical procedures) and generate or update the downstream model when the number of embeddings satisfies the threshold value. In this way, the downstream processing system 142 can include one or more embeddings in the known latent space (e.g., associated with one or more predetermined features of a given medical procedure or phase of a medical procedure) to enable the downstream model to be trained or updated to generate outputs for a specific medical procedure or procedures.

In some embodiments, the downstream model can be trained and implemented on edge devices to analyze data that is generated by the RMS 120 in real time. For example, the downstream model can be trained to obtain data associated with one or more frames generated during a surgical procedure and process the data in real-time. In this example, the downstream model can be trained based on the embeddings of the training dataset to allow for the implementation of a model that is reduced in size (e.g., includes fewer nodes or edges as the first model) and configured to receive the data associated with the one or more frames. This can allow for the implementation of downstream models that consume less processing and memory resources and, thus, are able to be implemented using resource-constricted edge devices (e.g., laptop computers, desktop computers) as opposed to less-constricted servers or cloud-computing devices.

With continued reference to FIG. 1, among others, the computing device 150 can include any combination of hardware or software that perform one or more of the functions described herein. For example, the computing device 150 can include any combination of hardware and software that receive and generate data associated with input received via the input device 154 and display a GUI via the display device 152. The computing device 150 can be the same as, or similar to, the computing device 500 of FIG. 5 or other computing devices described herein, and can include one or more tablets, laptops, desktops, servers, or virtual machines, or can be part of or include a cloud computing environment.

The computing device 150, or components thereof, can include a display device 152. The display device 152 can include any suitable display device such as a monitor, a touchscreen monitor, a liquid crystal display (LCD) monitor, a light emitting diode (LED) monitor. The computing device 150 can include an input device 154. The input device 154 can include any suitable input device 154 such as a keyboard, a mouse, a touchscreen, combinations thereof.

The computing device 150, or components thereof, can be located at least partially at the location of the surgical facility associated with the medical environment 110 or remotely therefrom. Elements of the computing device 150, or components thereof, can be accessible via portable devices such as laptops, mobile devices, wearable smart devices, etc. The computing device 150, or components thereof, can include other or additional elements that can be considered desirable to have in performing the functions described herein. The computing device 150, or components thereof, can include, or be associated with, one or more components or functionality of a computing including, for example, one or more processors coupled with memory that can store instructions, data or commands for implementing the functionalities of the computing device 150 discussed herein.

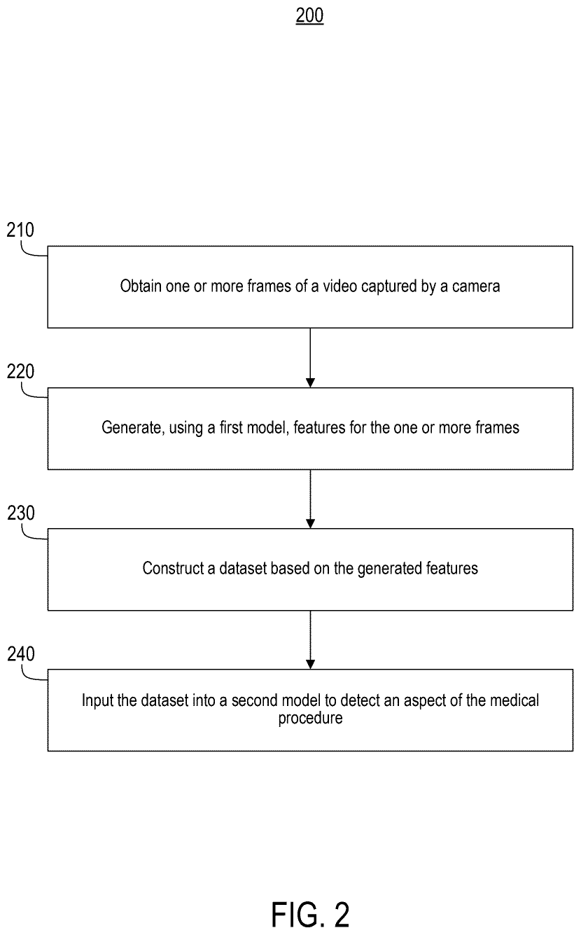

FIG. 2 is a flowchart diagram illustrating an example method 200 for extracting features to compress images generated during medical procedures, according to some embodiments. The method 200 can be performed by one or more systems, devices, or components depicted in FIG. 1, 4, or 5 including, for example, the data processing system 130 of FIG. 1.

At operation 210, a frame that captures a scene in a medical procedure is obtained. For example, a frame acquisition system can identify a frame or set of frames involved in a medical procedure. In this example, the frame or frames can include an image generated a visualization tool (e.g., a visualization tool that is the same as, or similar to, the visualization tool 116 of FIG. 1) when imaging one or more medical instruments (e.g., medical instruments that are the same as, or similar to, medical instruments 114 of FIG. 1) during the medical procedure. The frames can be included in a set forming a video captured by a camera of a medical procedure performed with a robotic medical system. The medical procedure can include a robot assisted surgery performed at least in part by a robotic medical system (e.g., an RMS that is the same as, or similar to, the RMS 120 of FIG. 1). In examples, the frames can be included in a video captured by a camera of a medical procedure performed with the RMS.

At operation 220, features for the one or more frames can be generated. For example, the data processing system can implement a feature generation system (e.g., that is the same as, or similar to, the feature generation system 138 of FIG. 1) to generate the features (represented by one or more embeddings). The feature generation system can implement one or more models that receive the frames as input and generate outputs representing embeddings. In examples, the embeddings can include a representation of one or more features of the frames (e.g., anatomical features illustrated by the frames) that include a dense numerical vector that captures its semantic meaning and relationships.

In examples, the one or more frames can be generated using a first model trained with self-supervised machine learning. For example, the feature generation system can provide the frames individual or in sets to the first model to cause the first model to generate outputs representing the features. In examples, the feature generation system can compare outputs of the first model with expected outputs to determine a difference. The feature generation system can then update one or more weights of the first model based on the difference. This process can be iteratively performed until the difference is reduced to below a threshold value (e.g., the model converges).

At operation 230, a dataset can be constructed. For example, a dataset construction system (e.g., that is the same as, or similar to, the dataset construction system 140 of FIG. 1) can construct the dataset based on the generated features. In examples, the dataset can include a plurality of embeddings corresponding to the plurality of frames for a given video representing a surgical procedure, a given portion of the video representing the surgical procedure.

In some embodiments, the dataset can be constructed to allow for training second models to perform specific tasks. For example, the dataset can be constructed such that the dataset includes frames associated with a given type of medical procedure or phase of medical procedure. In this example, the dataset can then be used to train second models to perform one or more tasks (e.g., segmentation) for a specific type of medical procedure.

At operation 240, the dataset can be input into a second model to detect an aspect of the medical procedure. For example, the dataset can be input into the second model to train the second model. In some examples, the dataset can be input into the second model to train the second model to identify features based on subsequently-input frames. The second model can include an untrained model (e.g., can be initialized with random variables and weights) or a trained model (e.g., a general model trained on a diverse dataset). In some examples, where the dataset is a trained model, the dataset can be input into the second model to fine-tune the second model to perform one or more tasks (e.g., segmentation of anatomical features commonly encountered during specific surgical procedures). In examples, at least portions of the dataset can be included with an input (e.g., one or more frames of a surgical procedure) as a few-shot prompt to condition the model and cause the model to detect the aspect of the medical procedure.

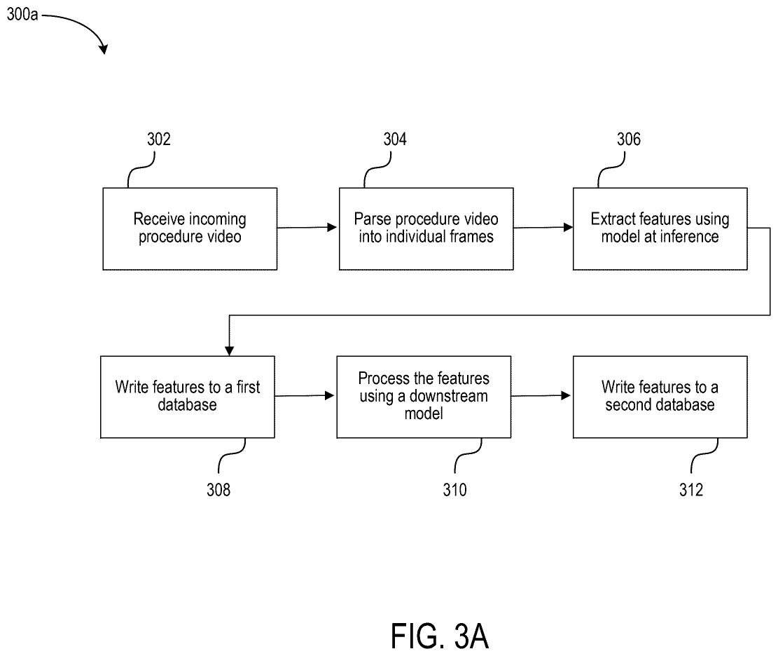

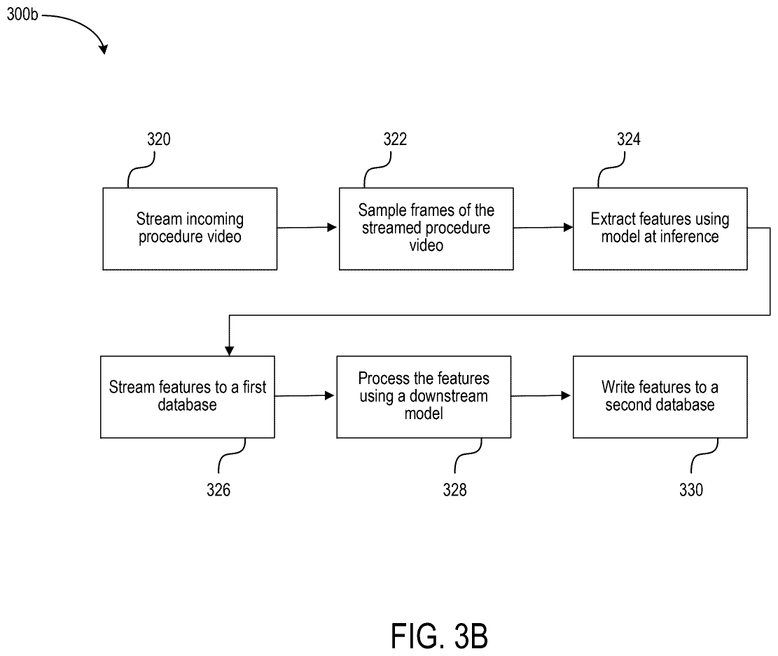

FIGS. 3A and 3B illustrate example processes for extracting features to compress images generated during medical procedures. The processes 300a and 300b can be performed by one or more systems, devices, or components depicted in FIG. 1, 4, or 5 including, for example, the data processing system 130 of FIG. 1.

The process 300a, at operation 302, includes receiving an incoming procedure video. The incoming procedure can include any surgical procedure involving the use of a robotic medical system (e.g., an RMS that is the same as, or similar to, the RMS 120 of FIG. 1). At operation 304, the video can be parsed into individual frames or portions of data associated with the channels for the frames. The individual frames or corresponding portions of data associated with the channels of the frames can be sampled at one or more predetermined rates (e.g., 1 fps, 2 fps) based on one or more aspects of the medical procedure (e.g., an anatomical structure of a medical procedure, a milestone of a medical procedure, a phase of a medical procedure, a task of a medical procedure).

At operation 306, features can be extracted from the frames. For example, a model (e.g., a vision transformer that is the same as, or similar to, the first model described with respect to FIG. 1) can receive the frames or data associated with the channels for the frames as input and generate an output including embeddings representing the features of each given frame. The embeddings can include vectors that represent the features present in a given frame as well as the semantic meaning associated with the features in a given frame or across multiple frames.

At operation 308, the features can then be written to a database (e.g., a data repository that is the same as, or similar to, the data repository 132 of FIG. 1). At operation 310 the features can be processed using a downstream model. For example, the features written to the database can be provided to a downstream model (e.g., another vision transformer, a CNN, an autoencoder, a U-net) to train or update the downstream model. In some examples, training can include adding one or more embeddings corresponding to a given feature with an input (e.g., a frame of another medical procedure) to allow the downstream model to perform a few-shot detection of the features in the input. At operation 312 the features output by the downstream model can be written to a second database. In this example, the second database can include a different dataset stored in a data repository.

The process 300b, at operation 320, includes receiving an incoming video stream of a procedure. The incoming video stream can represent any surgical procedure involving the use of the RMS or data associated with the channels for the frames. At operation 322, the frames of the video stream can be parsed into individual frames or corresponding portions of data associated with the channels of the frames. The individual frames can be sampled at one or more predetermined rates based on one or more aspects of the medical procedure. For example, the individual frames can be sampled at a predetermined rate based on a type of medical procedure, a phase of a medical procedure, etc., represented by the frames.

At operation 324, features can be extracted from the frames. For example, a model (e.g., a vision transformer that is the same as, or similar to, the first model described with respect to FIG. 1) can receive the frames or corresponding portions of data associated with the channels of the frames as input and generate an output including embeddings representing the features of each given frame. The embeddings can include vectors that represent the features present in a given frame as well as the semantic meaning associated with the features in a given frame or across multiple frames. For example, the embedding scan include vectors representing features such as visible portions of anatomical features that are present in each frame.

At operation 326, the features can then streamed to a database. At operation 328 the features can be processed using a downstream model. For example, the features written to the database can be provided to a downstream model as described herein to train or update the downstream model. At operation 330 the features output by the downstream model can be written to a second database. In this example, the second database can include a different dataset stored in a data repository.

FIG. 4 is a diagram of a medical environment 400, according to some embodiments. The medical environment 400 can refer to or include a surgical environment or surgical system. The medical environment 400 can include a robotic medical system 424 (e.g., a robotic medical system that is the same as, or similar to, the RMS 120 of FIG. 1), a user control system 410, and an auxiliary system 415 communicatively coupled one to another. A visualization tool 420 can be connected to the auxiliary system 415, which in turn can be connected to the robotic medical system 424. Thus, when the visualization tool 420 is connected to the auxiliary system 415 and this auxiliary system is connected to the robotic medical system 424, the visualization tool can be considered connected to the robotic medical system. The visualization tool 420 can be directly connected to the robotic medical system 424.

The medical environment 400 can be used to perform a computer-assisted medical procedure with a patient 425. A surgical team can include a surgeon 430A and additional medical personnel 430B-430D such as a medical assistant, nurse, and anesthesiologist, and other suitable team members who can assist with the surgical procedure or medical session. The medical session can include the surgical procedure being performed on the patient 425, as well as any pre-operative (e.g., which can include setup of the medical environment 400, including preparation of the patient 425 for the procedure), and post-operative (e.g., which can include clean up or post care of the patient), or other processes during the medical session. Although described in the context of a surgical procedure, the medical environment 400 can be implemented in a non-surgical procedure, or other types of medical procedures or diagnostics that can benefit from the accuracy and convenience of the surgical system.

The robotic medical system 424 can include a plurality of manipulator arms 435A-435D to which a plurality of medical instruments (e.g., the instruments described herein) can be coupled to, installed to, or supported by. The plurality of manipulator arms 435A-435D can include one or more linkages. Each medical instrument can be any suitable surgical tool (e.g., a tool having tissue-interaction functions), imaging device (e.g., an endoscope, an ultrasound tool, etc.), sensing instrument (e.g., a force-sensing surgical instrument), diagnostic instrument, or other suitable instrument that can be used for a computer-assisted surgical procedure on the patient 425 (e.g., by being at least partially inserted into the patient and manipulated to perform a computer-assisted surgical procedure on the patient). Although the robotic medical system 424 is shown as including four manipulator arms (e.g., the manipulator arms 435A-435D), in other embodiments, the robotic medical system can include greater than or fewer than four manipulator arms. Further, not all manipulator arms can have a medical instrument installed thereto at all times of the medical session. Moreover, a medical instrument installed on a manipulator arm can be replaced with another medical instrument as suitable.

One or more of the manipulator arms 435A-435D or the medical instruments attached to manipulator arms can include one or more displacement transducers, orientational sensors, positional sensors, or other types of sensors and devices to measure parameters or generate kinematics information. One or more components of the medical environment 400 can be configured to use the measured parameters or the kinematics information to track (e.g., determine poses of) or control the medical instruments, as well as anything connected to the medical instruments or the manipulator arms 435A-435D.

The user control system 410 can be used by the surgeon 430A to control (e.g., move) one or more of the manipulator arms 435A-435D or the medical instruments connected to the manipulator arms. To facilitate control of the manipulator arms 435A-435D and track progression of the medical session, the user control system 410 can include a display that can provide the surgeon 430A with imagery (e.g., high-definition 3D imagery) of a surgical site associated with the patient 425 as captured by a medical instrument installed to one of the manipulator arms 435A-435D. The user control system 410 can include a stereo viewer having two or more displays where stereoscopic images of a surgical site associated with the patient 425 and generated by a stereoscopic imaging system can be viewed by the surgeon 430A. The user control system 410 can also receive images from the auxiliary system 415 and the visualization tool 420.

The surgeon 430A can use the imagery displayed by the user control system 410 to perform one or more procedures with one or more medical instruments attached to the manipulator arms 435A-435D. To facilitate control of the manipulator arms 435A-435D or the medical instruments installed thereto, the user control system 410 can include a set of controls. These controls can be manipulated by the surgeon 430A to control movement of the manipulator arms 435A-435D or the medical instruments installed thereto. The controls can be configured to detect a wide variety of hand, wrist, and finger movements by the surgeon 430A to allow the surgeon to intuitively perform a procedure on the patient 425 using one or more medical instruments installed to the manipulator arms 435A-435D.

The auxiliary system 415 can include one or more computer systems (e.g., computing devices that are the same as, or similar to the computing device 500 of FIG. 5) configured to perform processing operations within the medical environment 400. For example, the one or more computer systems can control or coordinate operations performed by various other components (e.g., the robotic medical system 424, the user control system 410) of the medical environment 400. A computer systems included in the user control system 410 can transmit instructions to the robotic medical system 424 by way of the one or more computing devices of the auxiliary system 415. The auxiliary system 415 can receive and process image data representative of imagery captured by one or more imaging devices (e.g., medical instruments) attached to the robotic medical system 424, as well as other data stream sources received from the visualization tool. For example, one or more image capture devices can be located within the medical environment 400. These image capture devices can capture images from various viewpoints within the medical environment 400. These images (e.g., video streams) can be transmitted to the visualization tool 420, which can then passthrough those images to the auxiliary system 415 as a single combined data stream. The auxiliary system 415 can then transmit the single video stream (including any data stream received from the medical instrument(s) of the robotic medical system 424) to present on a display of the user control system 410.

The auxiliary system 415 can be configured to present visual content (e.g., the single combined data stream) to other team members (e.g., the medical personnel 430B-430D) who can not have access to the user control system 410. Thus, the auxiliary system 415 can include a display 640 configured to display one or more user interfaces, such as images of the surgical site, information associated with the patient 425 or the surgical procedure, or any other visual content (e.g., the single combined data stream). Display 440 can be a touchscreen display or include other features to allow the medical personnel 430B-430D to interact with the auxiliary system 415.

The robotic medical system 424, the user control system 410, and the auxiliary system 415 can be communicatively coupled one to another in any suitable manner. For example, the robotic medical system 424, the user control system 410, and the auxiliary system 415 can be communicatively coupled by way of control lines 445, which can represent any wired or wireless communication link as can serve a particular implementation. Thus, the robotic medical system 424, the user control system 410, and the auxiliary system 415 can each include one or more wired or wireless communication interfaces, such as one or more local area network interfaces, Wi-Fi network interfaces, cellular interfaces, etc.

It is to be understood that the medical environment 400 can include other or additional components or elements that can be needed or considered desirable to have for the medical session for which the surgical system is being used.

FIG. 5 is a block diagram depicting an architecture for a computing device 500 that can be employed to implement elements of the systems and methods described and illustrated herein, including aspects of the systems depicted in FIG. 1 or 4, and the methods depicted in FIGS. 2 and 3A-3B. For example, some or all of the components of the network 105, the medical environment 110, the RMS 120, the data processing system 130, the computing device 150, or the devices described with respect to medical environment 400 can include one or more component or functionality of computing device 500. The computing device 500 can be any computing device used herein and can include or be used to implement a data processing system or its components. The computing device 500 includes at least one bus 505 or other communication component or interface for communicating information between various elements of the computer system. The computer system further includes at least one processor 510 or processing circuit coupled to the bus 505 for processing information. The computing device 500 also includes at least one main memory 515, such as a random-access memory (RAM) or other dynamic storage device, coupled to the bus 505 for storing information, and instructions to be executed by the processor 510. The main memory 515 can be used for storing information during execution of instructions by the processor 510. The computing device 500 can further include at least one read only memory (ROM) 520 or other static storage device coupled to the bus 505 for storing static information and instructions for the processor 510. A storage device 525, such as a solid-state device, magnetic disk or optical disk, can be coupled to the bus 505 to persistently store information and instructions.

The computing device 500 can be coupled via the bus 505 to a display 530, such as a liquid crystal display, or active-matrix display, for displaying information. An input device 535, such as a keyboard or voice interface can be coupled to the bus 505 for communicating information and commands to the processor 510. The input device 535 can include a touch screen display (e.g., the display 530). The input device 535 can include sensors to detect gestures. The input device 535 can also include a cursor control, such as a mouse, a trackball, or cursor direction keys, for communicating direction information and command selections to the processor 510 and for controlling cursor movement on the display 530.

The processes, systems and methods described herein can be implemented by the computing device 500 in response to the processor 510 executing an arrangement of instructions contained in the main memory 515. Such instructions can be read into the main memory 515 from another computer-readable medium, such as the storage device 525. Execution of the arrangement of instructions contained in the main memory 515 causes the computing device 500 to perform the illustrative processes described herein. One or more processors in a multi-processing arrangement can also be employed to execute the instructions contained in the main memory 515. Hard-wired circuitry can be used in place of or in combination with software instructions together with the systems and methods described herein. Systems and methods described herein are not limited to any specific combination of hardware circuitry and software.

The processor 510 can execute one or more instructions associated with the system 100. The processor 510 can include an electronic processor, an integrated circuit including one or more of digital logic, analog logic, digital sensors, analog sensors, communication buses, volatile memory, nonvolatile memory. The processor 510 can include, but is not limited to, at least one microcontroller unit (MCU), microprocessor unit (MPU), central processing unit (CPU), graphics processing unit (GPU), physics processing unit (PPU), embedded controller (EC). The processor 510 can include, or be associated with, a main memory 515 operable to store or storing one or more non-transitory computer-readable instructions for operating components of the system 100 and operating components operably coupled to the processor 510. The one or more instructions can include at least one of firmware, software, hardware, operating systems, or embedded operating systems, for example. The processor 510 or the system 100 generally can include at least one communication bus controller to effect communication between the system processor and the other elements of the system 100.

The main memory 515 can include one or more hardware memory devices to store binary data, digital data. The main memory 515 can include one or more electrical components, electronic components, programmable electronic components, reprogrammable electronic components, integrated circuits, semiconductor devices, flip flops, arithmetic units. The main memory 515 can include at least one of a non-volatile memory device, a solid-state memory device, a flash memory device, a NAND memory device, a volatile memory device, etc. The main memory 515 can include one or more addressable memory regions disposed on one or more physical memory arrays.

Although an example computing system has been described in FIG. 5, the subject matter including the operations described in this specification can be implemented in other types of digital electronic circuitry, or in computer software, firmware, or hardware, including the structures disclosed in this specification and their structural equivalents, or in combinations of one or more of them.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

With respect to the use of plural or singular terms herein, those having skill in the art can translate from the plural to the singular or from the singular to the plural as is appropriate to the context or application. The various singular/plural permutations can be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description can illustrate a specific order of method steps, the order of such steps can differ from what is depicted and described, unless specified differently above. Also, two or more steps can be performed concurrently or with partial concurrence, unless specified differently above. Such variation can depend, for example, on the software and hardware systems chosen and on designer choice. All such variations are within the scope of the disclosure. Likewise, software implementations of the described methods can be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Some non-limiting embodiments of the present disclosure are described herein in connection with a threshold. As described herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold.

The foregoing description of illustrative implementations has been presented for purposes of illustration and of description. It is not intended to be exhaustive or limiting with respect to the precise form disclosed, and modifications and variations are possible in light of the above teachings or can be acquired from practice of the disclosed implementations. It is intended that the scope of the invention be defined by the claims appended hereto and their equivalents.

What is claimed is:

1. A system, comprising:
one or more processors, coupled with memory, to:
obtain one or more frames of a video captured by a camera of a medical procedure performed with a robotic medical system;
generate, using a first model trained with self-supervised machine learning, features for the one or more frames;
construct a dataset based on the generated features; and
input the dataset into a second model to detect an aspect of the medical procedure.

2. The system of claim 1, comprising the one or more processors to:
select the second model from a plurality of second models based on an attribute of the first model.

3. The system of claim 1, comprising the one or more processors to:
determine the first model is trained with self-supervised machine learning on a type of dataset; and
select the second model based on the second model being trained on the type of dataset.

4. The system of claim 1, comprising the one or more processors to:
select the first model configured to extract features based on a characteristic of the medical procedure.

5. The system of claim 1, comprising the one or more processors to:
select the second model configured to detect aspects of the medical procedure based on a characteristic of the medical procedure.

6. The system of claim 1, comprising the one or more processors to:
sample the video using a frame rate; and
select the one or more frames from the sampled video.

7. The system of claim 6, comprising the one or more processors to:
select the frame rate based on a characteristic of the first model or a characteristic of the second model.

8. The system of claim 6, comprising the one or more processors to:
receive, via an interface, a request to detect a type of aspect of the medical procedure; and
select the frame rate based on the type of aspect.

9. A system, comprising:
one or more processors, coupled with memory, to:
obtain data associated with a set of images generated by at least one camera during a medical procedure;
sample the set of images to generate a sampled set of images;
provide an image of the sampled set of images as input to a model to cause the model to generate an output comprising one or more embeddings that represent one or more features, the one or more features corresponding to aspects of medical procedures in a latent space;
generate a dataset based on the one or more embeddings in the latent space; and
provide a portion of the dataset to a downstream model to cause the downstream model to generate an output, the output of the downstream model based on the portion of the dataset.

10. The system of claim 9, comprising the one or more processors to:
obtain the data associated with the set of images from at least one sensor supported by a robotic surgical system.

11. The system of claim 9, comprising the one or more processors to:
provide the image of the sampled set of images as input to the model, the model comprising one or more layers associated with an attention function.

12. The system of claim 9, comprising the one or more processors to:
provide the image of the sampled set of images as input to the model, the model trained based on one or more operations performed by the model and a second model.

13. The system of claim 12, wherein the one or more operations performed by the model and the second model comprise:
augmenting at least one training image associated with a training dataset comprising images generated during training procedures to generate a first augmented image and a second augmented image, the at least one training procedure comprising a medical procedure that is different from the at least one medical procedure.

14. The system of claim 13, wherein the one or more operations performed by the model and the second model comprise:
providing the first augmented image to the model to cause the model to generate a first training output;
providing the second augmented image to the second model to cause the second model to generate a second training output;
determining a loss based on a difference between the first training output and the second training output; and
updating weights of the model or the second model based on the loss.

15. A method, comprising:
obtaining, by one or more processors coupled with memory, one or more frames of a video captured by a camera of a medical procedure performed with a robotic medical system;
generating, by the one or more processors, using a first model trained with self-supervised machine learning, features for the one or more frames;
constructing, by the one or more processors, a dataset based on the generated features; and
inputting, by the one or more processors, the dataset into a second model to detect an aspect of the medical procedure.

16. The method of claim 15, comprising:
selecting the second model from a plurality of second models based on an attribute of the first model.

17. The method of claim 15, comprising:
determining the first model is trained with self-supervised machine learning on a type of dataset; and
selecting the second model based on the second model being trained on the type of dataset.

18. The method of claim 15, comprising:
selecting the first model configured to extract features based on a characteristic of the medical procedure.

19. The method of claim 15, comprising:
selecting the second model configured to detect aspects of the medical procedure based on a characteristic of the medical procedure.

20. The method of claim 15, comprising:
sampling the video using a frame rate; and
selecting the one or more frames from the sampled video.
